(19) United States
(12) Patent Application Publication
(10) Pub. No.: US 2024/0363248 A1
(43) Pub. Date: Oct. 31, 2024

(54) NEURAL-NETWORK-BASED CLASSIFIER

(71) Applicant: CRAIF INC., Tokyo (JP)

(72) Inventor: Yuting CHEN, Aichi (JP)

(21) Appl. No.: 18/685,689

(22) PCT Filed: Aug. 22, 2022
(86) PCT No.: PCT/JP2022/031477

§ 371 Date: Feb. 22, 2024

(30) Foreign Application Priority Data
Aug. 25, 2021 (JP) 2021-137228

(51) Int. Cl. G16H 50/20; G16H 10/40

(52) U.S. Cl. CPC G16H 50/20; G16H 10/40

(57) ABSTRACT

There is provided a method of training a classifier to predict a disease status of a patient, to be executed by a processor, the method comprising: i) receiving a plurality of training data sets derived from a plurality of batches, each training data set comprising: a diagnostic data; a batch ID; and a disease ID; ii) using the received plurality of training data sets to generate a batch classifier; and iii) using the received plurality of training data and the generated batch classifier to generate a disease status classifier.

### TECHNICAL FIELD

The disclosure herein generally relates to methods, processes and systems for classifying data including explanatory variables confounding factors using a neural network, and in one example, to methods, processes and systems for classifying the likelihood of a biological subject having at least one medical condition. However, the claimed inventions and the present disclosure should not be construed in a limiting manner due to the description of this section.

For example, in the field of disease diagnosis, a neural network classifier trained by machine learning can be used to diagnose a subject's disease from measurements obtained by biological techniques. The genetic information of the subject obtained by DNA sequencing, the expression information of each biomolecule using DNA/protein/peptide microarray, etc. are acquired, and the acquired information is input to a pre-trained neural network classifier. By doing so, it is possible to make an appropriate diagnosis of the subject or obtain a result useful for the diagnosis.

Training data are used to train neural networks and generate appropriate classifiers. Such training data may be collected from multiple batches. However, statistical analysis based on data collected from multiple batches can face the so-called batch effect. Batch effect represents the systematic technical differences when samples are processed and measured in different batches and which are unrelated to any biological variation recorded during the microarray gene expression experiment (non-patent literature 1). In the presence of confounding factors such as batch effect, the neural network model cannot be trained to generate an appropriate classifier.

### SUMMARY OF INVENTION

A non-limiting object of the present disclosure is to use a neural network classifier generated using training data in which confounding factors may be present and to classify input information that may be affected by confounding factors.

In one aspect, a method of training a classifier that predicts the objective variable of interest is provided. The method may be performed by a computer. The method may comprise at least a part of the neural network. The method may comprise training data from multiple batches, using the training data to generate a batch classifier, and using the training data and a batch classifier to generate an objective variable classifier. The training data may include explanatory variables, objective variables, and batch information.

In one aspect, a method of predicting the objective variable of interest is provided. The method may be performed by a computer. An objective variable classifier may be provided that includes a neural network as at least a portion thereof. The method may comprise providing a trained objective variable classifier; acquiring target data including explanatory variables and batch information; using the objective variable classifier to perform batch classification of the acquired target data; predicting the objective variable of the target data based on the batch classification and the explanatory variables of the obtained target data.

### BRIEF DESCRIPTION OF DRAWINGS

FIG. 1 illustrates a structure of a neural-network-based classifier according to an embodiment.

FIG. 2 illustrates a structure of a neural-network-based classifier according to an embodiment.

FIG. 3 illustrates a structure of a neural-network-based classifier according to an embodiment.

FIG. 4 shows confusion matrices as a result of a training of a batch classifier.

FIG. 5 illustrates a structure of a neural-network-based classifier for training.

FIG. 6 shows confusion matrices as a result of a training of the neural-network-based classifier according to an example.

FIG. 7 shows ROC curves (A) and Precision-Recall curves (B) as a result of a training of the neural-network-based classifier according to an example.

FIG. 8 shows confusion matrices as a result of a training of the neural-network-based classifier according to a comparative example.

FIG. 9 shows ROC curves (A) and Precision-Recall curves (B) as a result of a training of the neural-network-based classifier according to a comparative example.

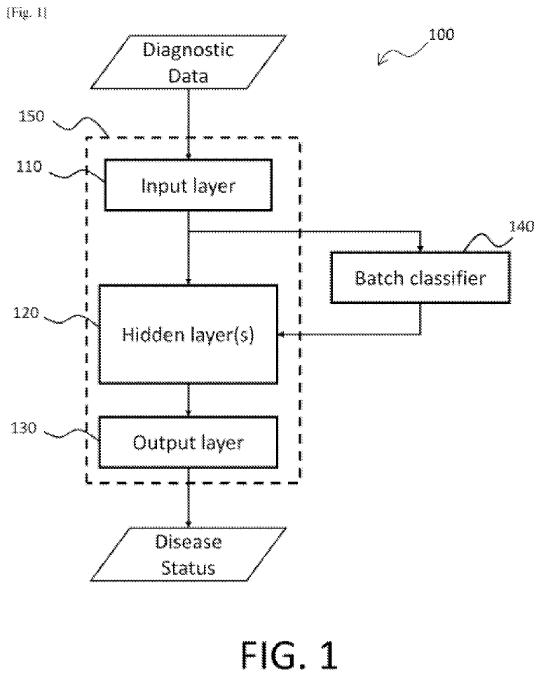

In one aspect, a system is provided that predicts or classifies the objective variable of interest. FIG. 1 illustrates a configuration of a system (classifier 100) according to an embodiment. The classifier 100 includes a main neural network 150, and a batch classifier 140.

The main neural network 150 includes an input layer 110, one or more hidden layers 120, and an output layer 130. The batch classifier 140 receives signals (hereinafter also referred to as data) from the input layer 110 and predicts the batch ID of the input data. The predicted batch ID is input to the hidden layer 120 of the main neural network 110. The main neural network 110 includes the objective variable of the input data or classifies the input data based on the preprocessed input data from the input layer 110 and the predicted batch ID from the batch classifier 140. The output layer 130 outputs the classified result.

The classifier 100 may include a preprocessing device as an algorithm or device that converts or processes input data including explanatory variables into a data format suitable for the next processing (not shown). Such a pretreatment unit may not be included in the classifier 100 and may be configured to be connected to the classifier 100.

As used herein, the term “classifier” generally refers to an algorithm or software that statistically classifies data into multiple groups.

As used herein, the term “neural network classifier” generally refers to a classifier that includes an artificial neural network, at least in part.

As used herein, the term “neural network” generally refers to a mathematical model (artificial neural network, ANN) composed of artificial neurons (nodes) interconnected by artificial synapses. The neural network may have one or more layers. A neural network may be a non-hierarchical type in a part thereof. A layer may be defined as a set of nodes. A layer may be defined to consist of a tensor-in tensor-out computation function (the layer's call method) and some state, held in TensorFlow variables (the layer's weights), for example not in a limiting manner, in accordance with Keras.

The neural network may include an input layer that accepts external data. A plurality of input layers may be arranged. The neural network may include an output layer that produces or outputs the result. At least one or more hidden layers (intermediate layers) may be provided between them.

A layer may be, for example in accordance with the definition of Keras, but not limited to, a core layer, such as Input object, Dense layer, Activation layer, Embedding layer, Masking layer and Lambda layer; a convolution layer; a pooling layer; a recurrent layer; a preprocessing layer; a normalization layer; a regularization layer; an attention layer; a reshaping layer; and a merging layer such as a concatenation layer.

In some embodiments, the neural network may be, for example without limitation, Perceptron (P), Feed Forward (FF), Radial Basis Network (RBF), Deep Feed Forward (DFF), Recurrent Neural Network (RNN), Long/Short Term Memory (LSTM), Gated Recurrent Unit (GRU), Auto Encoder (AE), Variational AE (VAE), Denoising Auto Encoder (DAE), Sparse AE (SAE), Markov Chain (MC), Hopfield Network (HN), Boltzmann Machine (BM), Restricted BM (RBM), Deep Belief Network (DBN), Deep Convolutional Network (DCN), Deconvolutional Network (DN), Deep Convolutional Inverse Graphics Network (DCIGN), Generative Adversarial Network (GAN), Liquid State Machine (LSM), Extreme Learning Machine (ELM), Echo State Network (ESN), Deep Residual Network (DRN), Kohonen Network (KN), and Neural Turing Machine (NTM).

In some embodiments, the “neural network” may include a “batch classifier” as part thereof, or may be combined with an externally located “batch classifier”. Even when the “neural network” includes a “batch classifier”, the output of the “batch classifier” can be considered to be a “hidden layer” of the “neural network”. In that case, the “neural network” is configured to have a portion of the substantive (or main) neural network and a batch classifier, meaning that the output of the batch classifier is input to that substantive neural network. In the present specification, both the “substantial neural network” or “main neural network” and the entire configuration including the batch classifier may be referred to as a “neural network” or “neural network classifier”.

As used herein, the term “batch classifier” refers to an algorithm that predicts a “batch” that matches the target variable based on the input target variable. The batch classifier predicting batches may include selecting from a group of batches containing a finite number of batches previously formed, for example by training. The batch classifier may generate a new batch which is not previously prepared, according to the input target variable and output the generated batch to the input target variable.

In some embodiments, the batch classifier may not include a so-called neural network. In some embodiments, the batch classifier may use a regression model. The regression model may be a linear regression model. The linear regression model may be, for example without limitation, a general linear model, a generalized linear model, or the like. In some embodiments, the batch classifier may use a logistics regression model.

In some embodiments, the batch classifier may use a non-linear regression model. The nonlinear regression model may be, for example without limitation, non-limitingly k-nearest neighbor method, classification tree, random forest, neural network, support vector regression, projection pursuit regression, and the like. In some embodiments, the batch classifier may include a neural network.

The explanatory variables and batch information may not be input to the same input layer. In some embodiments, the output of the batch classifier may be input to one of the hidden layers of the substantive neural network. For example, the explanatory variables may be input to the input layer of the neural network, the batch information may be input to another batch classifier, and the output of the batch classifier may be input to one of the hidden layers of the neural network.

The neural network may include one or more hidden layers, or at least one hidden layer. The hidden layer may include a plurality of hidden layers.

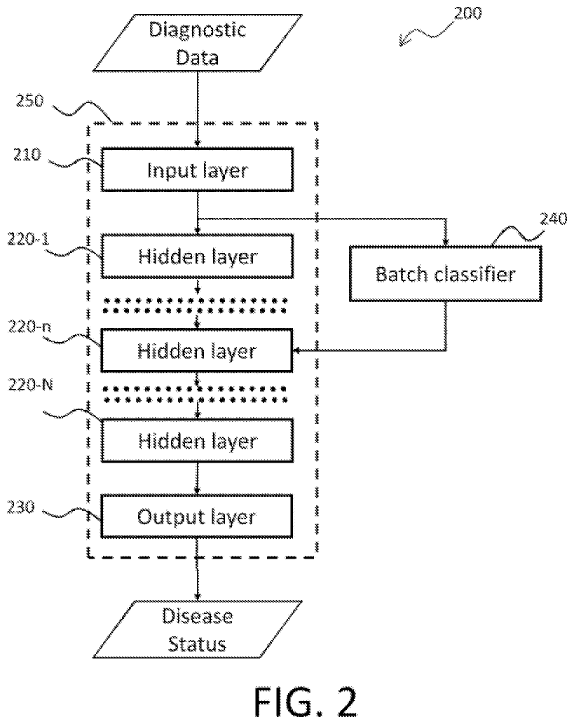

The output of the batch classifier may be input to one of a plurality of hidden layers. For example, as illustrated in FIG. 2, a neural network classifier 200 may include a main neural network 250 and a batch classifier 240. The main neural network 250 includes an input layer 210, N hidden layers 220-1˜220-N, and an output layer 230. The batch classifier 240 receives data from the input layer 210 and predicts the batch ID of the input data. The predicted batch ID is input to a hidden layer 220-n which is one of the plurality of the N hidden layers.

In some embodiments, the output of the batch classifier may be input to one of the middle or second half layers (n>or □N/2) of the plurality of hidden layers. For example, if there are 3 hidden layers, the output of the batch classifier may be input to the second hidden layer or the third hidden layer. For example, if there are 4 hidden layers, the output of the batch classifier may be input to the third hidden layer or the fourth hidden layer. (In some aspects, the “second half” refers to the one closer to the output of the neural network and may also be called the “last”. In some aspects, the “first half” refers to the one closer to the input of the neural network and is also called the “first”.)

In some embodiments, the output of the batch classifier may be input to one of the latter third of the plurality of hidden layers (n>or □N/3). In some embodiments, the output of the batch classifier may be input to one of the last quarter of the plurality of hidden layers (n>or □N/4). The output of the batch classifier may be input to one of the latter half N (N=integer) of the plurality of hidden layers. In some embodiments, the output of the batch classifier may be input to the last layer of the plurality of hidden layers or the hidden layer immediately preceding the output layer.

By inputting the output of the batch classifier in the middle or the latter half of the hidden layer, the influence of the batch classification on the final output of the neural network can be strengthened, for example in a non-limiting manner.

In some embodiments, the output of the batch classifier may be input to one of the middle or first half layers of the plurality of hidden layers. In some embodiments, the output of the batch classifier may be input to one of the first thirds of the plurality of hidden layers. In some embodiments, the output of the batch classifier may be input to one of the first quarters of the plurality of hidden layers. In some embodiments, the output of the batch classifier may be input to one of the first half (n<or □N/2) of the plurality of hidden layers. In some embodiments, the output of the batch classifier may be input to the first layer of multiple hidden layers or to the hidden layer immediately after the input layer.

By inputting the output of the batch classifier in the middle or the first half of the hidden layer, the effect of batch classification on the final output of the neural network can be strengthened but less than if it is put into the input layer, for example in a non-limiting manner.

In some embodiments, the output of the batch classifier may be input to one of the hidden layers between n1-th hidden layer and n2-th hidden layer (1□n1<n2≤N). Such a hidden layer “n” or the range of the hidden layers such as n1, n2 may be expressed as n1 or n2=N* (p/q) or N/r, where p, q and r are integers. If n, n1 or n2=N* (p/q) or N/r is not an integer, it can be replaced or calculated by rounding up or down, or rounding to the nearest whole number.

As used herein, the term “explanatory variables” are used interchangeably with predictors and independent variables, unless otherwise specified.

As used herein, the term “objective variable” is used interchangeably with response variables, result variables, dependent variables, and reference variables, unless otherwise specified.

In some embodiments, the explanatory variables may be diagnostic data or data associated with the diagnostic data, and the objective variable may be disease, medical condition or data related thereto.

In some embodiments, the diagnostic data may include the medical history of a subject (e.g., a patient), the results of clinical and medical tests, and/or inferences of a physician or medical diagnostic system based on them.

In some embodiments, the diagnostic data may be derived from a clinical examination (hereinafter also referred to as “laboratory test”). The clinical examination include, without limitation, specimen tests, biopsy, diagnostic imaging, pathological diagnosis, physical tests, psychological tests, and other tests aimed at or without the presence or absence of illness to obtain relevant information.

The specimen tests include, without limitation, biochemical tests, hematological tests, urine/feces tests, immunological tests, microbiological tests and the like.

In the present disclosure, the body fluid used for the test means a body fluid obtained from the subject or a sample derived from the body fluid. The body fluid may be, but is not limited to, blood, serum, plasma, lymph, tissue fluid such as interstitial fluid, intercellular fluid, intracellular fluid, and cerebrospinal fluid, cerebrospinal fluid, and cerebrospinal fluid. It may be abdominal fluid, plasma sac fluid, cerebrospinal fluid (cerebrospinal fluid), joint fluid (slip fluid), or interstitial fluid (aqueous humor). The body fluid may be digestive juice such as saliva, gastric juice, bile, pancreatic juice, and intestinal juice, and may be sweat, tears, nasal mucus, urine, semen, vaginal juice, amniotic fluid, and milk. The body fluid may be an animal body fluid or a human body fluid.

“Biopsy” includes, without limitation, respiratory and circulatory function tests, ultrasonography, various tests using monitoring devices, brain wave tests, nerve/muscle tests, otolaryngology tests, ophthalmic tests, dermatological tests, clinical psychology/neuropsychiatric tests, examinations using radioisotopes, endoscopy and the like. In some embodiments, the biopsy may be a liquid biopsy. In some embodiments, the biopsy may be performed on tissue obtained during surgery.

In some embodiments, the diagnostic data may be an amount, frequency or other test values associated with gene expression in a genetic test. Diagnostic data may include, for example, gene expression levels.

The gene may be a nucleic acid (at least one of DNA and RNA). RNA may be messenger RNA (mRNA), transfer RNA (tRNA), liposome RNA (rRNA), microRNA (miRNA), or the like. DNA and RNA may be cell-free DNA/RNA (cfDNA/RNA), intracellular DNA/RNA, intracellular DNA/RNA, and the like. DNA/RNA may be obtained by liquid biopsy.

In the genetic test, the body fluid (blood, saliva, urine, etc.) of the subject may be obtained and the amount of a predetermined or arbitrary nucleic acid (relative amount, absolute amount) may be measured. Nucleic acid may be amplified. Nucleic acid may be measured using a genetic analysis device such as a DNA chip (also called a microarray) or a sequencer.

Genetic testing may include testing for genetic displacement. Copy number changes may be measured as gene displacements. Changes in the number and expression level of single nucleotide polymorphisms (SNPs) may be measured. The fusion gene may be measured. For example, it may be determined whether or not fusion occurs at a predetermined gene or base site. The number of fusion genes may be measured. Chromosomal abnormalities may be measured. The presence or absence of chromosomal abnormalities, the amount or frequency within the predetermined region, and the like may be measured. Chromosomal abnormalities may be structural changes, changes in the number of chromosomes, or both. Tumor gene displacement (TMB) may be measured. The number of tumor genes or TMG score may be measured. The amount of epigenetic changes such as methylation (number of sites, frequency at a given site), acetylation, etc. may be measured. The number of sites where these displacements occur or the amount of change at a predetermined site may be measured. Microsatellite instability (MSI) analysis or testing may be performed. The number or frequency of altered bases in the microsatellite region may be measured. Splicing anomalies may be measured. The presence or absence of the abnormality may be measured, and the number (number or number of bases), absolute number, frequency, etc. of the location may be measured.

In some embodiments, the explanatory variables may be morphological data. The term “morphologics” in the field of biology generally refers to a comprehensive identification method for features related to the shape of living things, organs of living things, and so on.

The explanatory variables may include, for example, image data such as a microscope image such as a laser microscope, a fluorescence microscope, an electron microscope, and an atomic force microscope (AFM), an X-ray image, a CT image, and an ultrasonic image. The biological image may be a micrograph of an organ, a cell, or the like.

In some embodiments, the explanatory variables may be “omics” data. The term “omics” generally refers to a substance or concept represented by the suffix “ome”. Omics data includes, for example without limitation, data related to genome, transcriptome, proteome, glycome, lipidome, metabolome, physiome, phenome, biome and the like.

In some embodiments, the diagnostic data may include RNA expression levels. RNA expression levels may be obtained, for example, without limitation, by techniques such as microarray measurement, sequencing, and electrophoresis. RNA expression levels may include expression levels of a plurality of RNAs and may include RNA expression profiles.

In some embodiments, the classifier may be used to diagnose and/or prognose a subject's disease, or to do and/or help any type of medical decision, prediction and/or observation. The disease may be a cancer. The cancer includes as example, but is not limited to, brain tumor, lung cancer, breast cancer, thyroid cancer, esophagus cancer, liver cancer, biliary tract cancer, gastric cancer, pancreas cancer, colorectal cancer, prostate cancer, renal cancer, bladder cancer, uterine cancer, cervical cancer, ovarian cancer, skin cancer, lymphoma, leukemia.

### Example 1

As an Example, miRNA expression data was used to train a neural network classifier, and the classifier was used to predict the disease status of unknown subjects (patients), or classify the disease status to either one of cancerous and non-cancerous.

### 1-1. Neural Network Model

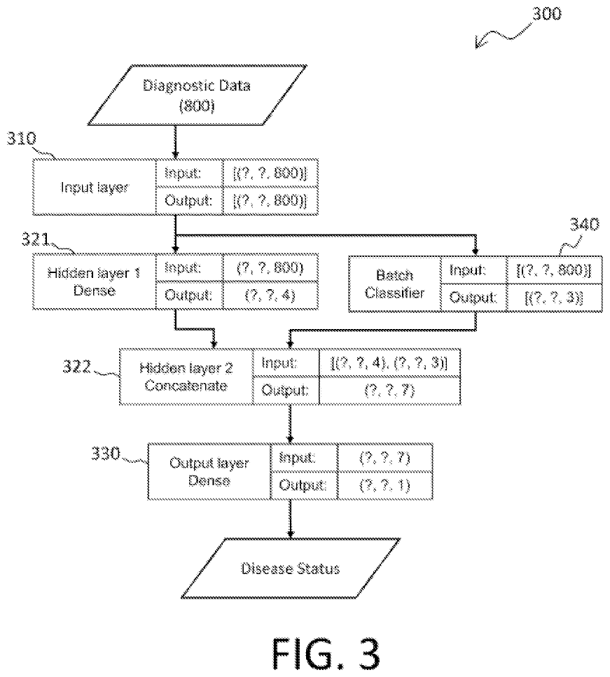

FIG. 3 illustrates the structure of a classifier 300 used for this Example. This classifier 300 for the present Example, but is not limited thereto and can be applied to other embodiments and examples. This classifier has an input layer 310, intermediate layers (the first intermediate layer/hidden layer/dense layer 321, and the second intermediate layer/concatenation layer 322), a batch classifier 340, and an output layer 330.

The input layer 310 can receive 800 dimensions of input data related to the diagnostic data. In the present Example in particular, these 800 inputs correspond to the 800 miRNA expression levels, respectively. The input layer 310 outputs the data related to 800 miRNA expression levels to the first intermediate layer 321 and to the batch classifier 340.

The first intermediate layer 321 is configured to function as a dense layer. The first intermediate layer 321 receives 800 dimensions of data from the input layer 310 and outputs 4 dimensions of data to the second intermediate layer 322.

The batch classifier 340 receives the 800 dimensions of data from the input layer 310, predicts the batch ID of the input data, and outputs 3 dimensions of data to the second intermediate layer 322. The batch classifier 340 in FIG. 3 outputs 3 dimensions of data. But the dimension of the output data from the batch classifier is not limited thereto and can be another dimension.

The second intermediate layer 330 is configured to function as a concatenation layer. The concatenation layer 322 receives the 4-dimensional output from the dense layer 321 and the 3-dimensional output from the batch classifier 340 and concatenates them to a 7-dimensional data as output to the output layer 330.

The output layer 330 is configured to function as a dense layer. The output layer 330 outputs the disease status. This output is the prediction result of the entire classifier system 300 with the diagnostic data of 800 miRNA levels.

### 1-2 Samples

In total 102 urine samples were collected from three projects, or cohorts, PJ001, PJ002, PJ003. Among them, 25 non-cancers and 21 ovarian cancers were from PJ001 project; ovarian cancers were from PJ002 project; and 15 non-cancers were from PJ003 project. Thus, the samples were collected in a very unbalanced way across different projects.

### 1-3 Data Acquisition

miRNA expression levels were measured by using TORAY 3D-Gene microarray. After performing the quality control and normalization, 800 miRNAs with the highest average expression levels were selected out of several thousand miRNA's, to be used for the classification. The entire data set from 102 samples were split into 81 samples (80%) for training and validation, and 21 samples (20%) for hold-out testing.

### 1-4 Training of Neural Network Based Classifier

### 1-4-1 Training of Batch Classifier

A logistic regression model was adopted for training the batch classifier 340. This model was trained and validated using the training set. The leave-one-out cross validation (LOOCV) was used to tune the hyperparameter C which is the L1 regularization strength.

The units were tuned for 4 and 5, resulting in the optimized value of 4. The hyperparameter C (L1) was tuned between 0.001 and 10, resulting in the optimized value of C=0.001.

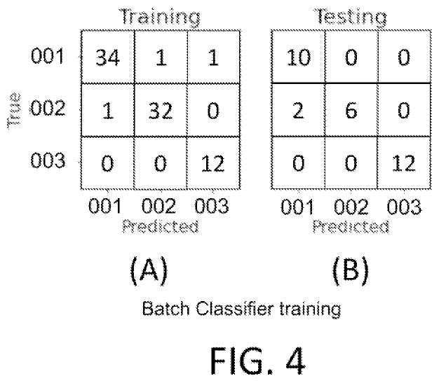

The derived model was evaluated on the hold-out testing dataset. FIG. 4(A) shows a confusion matrix of the training result. FIG. 4(B) shows a confusion matrix of the testing result. The training result shows the batch classifier can correctly predict batch labels in both training and testing datasets, with overall accuracy being 0.96 and 0.93.

### 1-4-2 Training of Main Neural Network

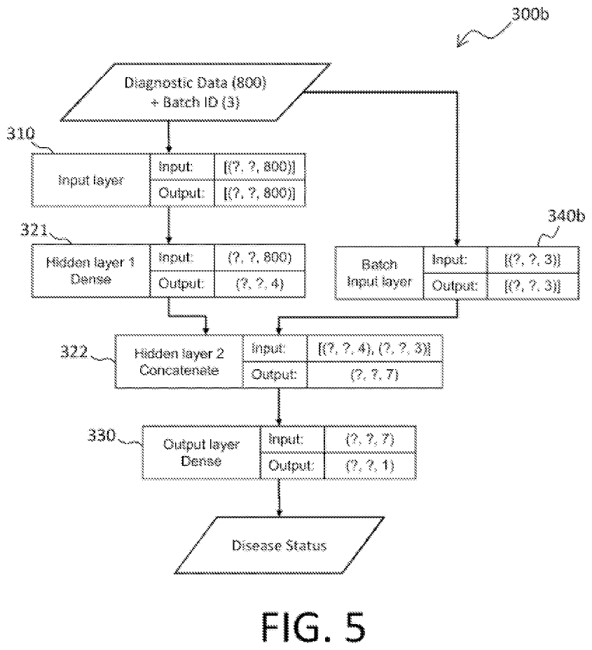

FIG. 5 illustrates a visualization of the network, or network graph of the neural network 300b, to train the main neural network including the input layer 310, the first and second intermediate layers 321,322 and the output layer 330 which are shown in FIG. 4. The first layer 321 is a “dense” layer as a hidden layer. The second layer 322 is a concatenation layer. In the neural network 300b that undergoes training, the batch classification 340 is not disposed. Instead of the batch input layer 340b is disposed.

The training data of 81 samples (80%) were used together with their batch IDs (or project/cohort code). The 800 dimensions of diagnostic data were input in the input layer 310. The 3 dimensions of batch ID were input in the batch input layer 340b. The batch input layer 340b output the batch ID data to the concatenation layer 322. In this way, the concatenation layer 322 received the correct answer in terms of the batch ID. Thus, the concatenation layer 322 received the 4-dimensional output from the dense layer 321 and the 3-dimensional output from the batch input layer 340b and concatenated them to a 7-dimensional data as output to the output layer 330. In this way, the main neural network portion has been trained.

### 1-4-3 Validation of Trained Classifier

The batch classifier and the main neural network, which have both been trained as explained above, were combined to form a classifier to predict disease status using the 800 miRNA levels as input.

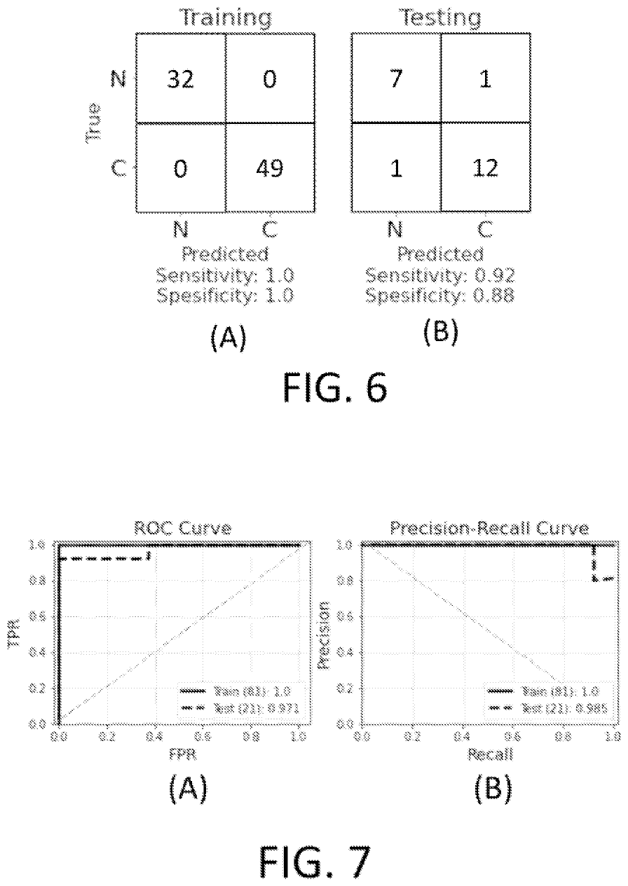

The trained entire classifier 300 as shown in FIG. 3 was evaluated by using the hold-out testing dataset. FIG. 6(A) and FIG. 6(B) show confusion matrices of the training result and the testing result, respectively. “N” stands for non-cancerous and “C” stands for cancerous. The testing result shows sensitivity of 0.92 and specificity of 0.88.

FIG. 7 shows ROC curves (A) and Precision-Recall curves (B) for the training set (broken line) and the test set (solid line). ROC curves in FIG. 7. (A) are represented by FPR and TPR. Precision-Recall curves in FIG. 7. (B) are represented by “Recall” and “Precision”. “Recall” is defined by TPR/(TPR+FNR) and “Precision” is defined by TPR/(TPR+FPR). Here, TPR, FPR, FNR stand for true positive rate, false positive rate, and false negative rate, respectively. AUC (Area Under the Curve) of the test set was 0.981 and 0.962 for the ROC curve (FIG. 7(A)) and the Precision-Recall curve (FIG. 7(B)), respectively.

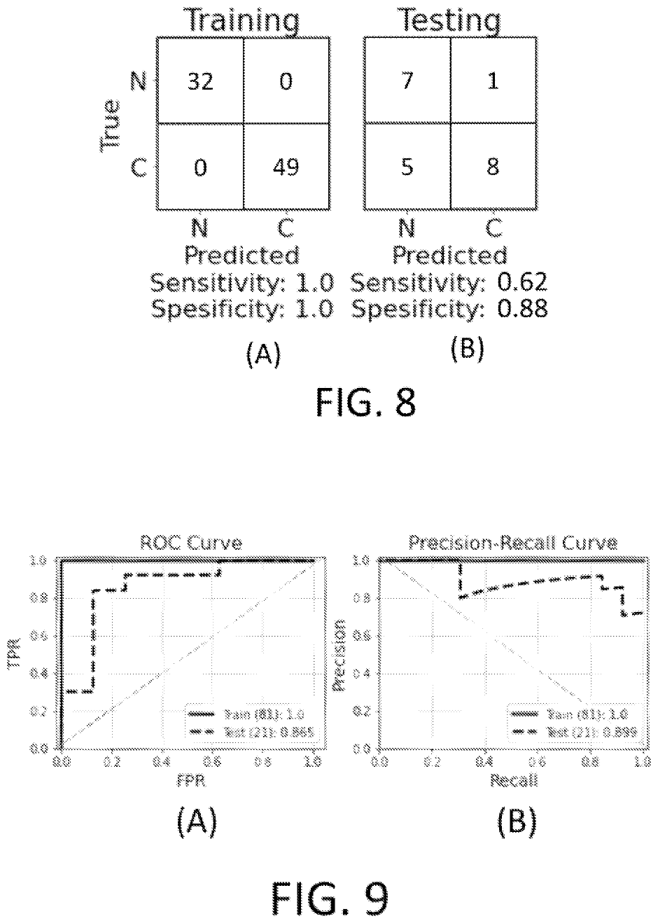

As a comparison, a neural network without a batch classifier is trained and tested. By referring to FIG. 3, this neural network model has a structure having an input layer 310, one hidden layer 321 and an output layer 330, but does not have a batch classifier 340. FIG. 8(A) and FIG. 8(B) show confusion matrices of the training result and the testing result, 14 respectively. As shown in FIG. (8), the testing result shows sensitivity of 0.62 and specificity of 0.88. FIG. 9 shows Precision-Recall curves (A) and ROC curves (B) for the training set (broken line) and the test set (solid line). AUC of the test set was 0.865 and 0.899 for the ROC curve (FIG. 9(A)) and the Precision-Recall curve (FIG. 9(B)), respectively.

Thus, the neural network model with a batch classifier showed better results in sensitivity, specificity and AUC of Precision-Recall curve and ROC curves. In other words, it was trained to have a higher performance, than the corresponding neural network model without a batch classifier.

The present disclosure also includes, but not limited to, the following embodiments.

A001. A method of training a classifier to predict a disease status of a patient, to be executed by a processor, the method comprising:
i) receiving a plurality of training data sets derived from a plurality of batches, each training data set comprising: a diagnostic data; a batch ID; and a disease ID;
ii) using the received plurality of training data sets to generate a batch classifier; and
iii) using the received plurality of training data and the generated batch classifier to generate a disease status classifier.

A002. A method of training a classifier to predict a responsible variable of a subject, to be executed by a processor, the method comprising:
i) receiving a plurality of training data sets derived from a plurality of batches, each training data set comprising: an (one or more) explanatory variable; a batch ID; and a target variable; and
ii) using the received plurality of training data sets to generate a batch classifier; and
iii) using the received plurality of training data and the generated batch classifier to generate a target variable classifier.

A011. The method of A001 or A002 or any one of the embodiments,
wherein said generating the disease status classifier comprises using a neural network.

A012. The method of A011 or any one of the embodiments,
wherein the neural network comprises an input layer, at least one hidden layers and an output layer, and
wherein step iii) comprises inputting an output of the batch classifier in one of the at least one hidden layers.

A013. The method of A011 or any one of the embodiments,
wherein the neural network comprises an input layer, a plurality of hidden layers and an output layer, and
wherein step iii) comprises inputting an output of the batch classifier in one of the plurality of hidden layers.

A014. The method of A013 or any one of the embodiments,
wherein step iii) comprises inputting an output of the batch classifier in one of the second half from the middle of the plurality of hidden layers.

A015. The method of A013 or any one of the embodiments,
wherein step iii) comprises inputting an output of the batch classifier in one of the last third of the plurality of hidden layers.

A016. The method of A013 or any one of the embodiments,
wherein step iii) comprises inputting an output of the batch classifier in one of the last quarter of the plurality of hidden layers.

A017. The method of A013 or any one of the embodiments,
wherein step iii) comprises inputting an output of the batch classifier in the last hidden layer just before the output layer.

A021. The method of any one of A011 to A017 or any one of the embodiments,
wherein the neural network is selected from the group consisting of: Perceptron (P), Feed Forward (FF), Radial Basis Network (RBF), Deep Feed Forward (DFF), Recurrent Neural Network (RNN), Long/Short Term Memory (LSTM), Gated Recurrent Unit (GRU), Auto Encoder (AE), Variational AE (VAE), Denoising Auto Encoder (DAE), Sparse AE (SAE), Markov Chain (MC), Hopfield Network (HN), Boltzmann Machine (BM), Restricted BM (RBM), Deep Belief Network (DBN), Deep Convolutional Network (DCN), Deconvolutional Network (DN), Deep Convolutional Inverse Graphics Network (DCIGN), Generative Adversarial Network (GAN), Liquid State Machine (LSM), Extreme Learning Machine 16 (ELM), Echo State Network (ESN), Deep Residual Network (DRN), Kohonen Network (KN), Support Vector Machine (SVM), and Neural Turing Machine (NTM).

A031. The method of any one of A001 to A021 or any one of the embodiments,
wherein step ii) comprises training the batch classifier.

A032. The method of A031 or any one of the embodiments,
wherein said training the batch classifier comprises using a regression model.

A033. The method of A032 or any one of the embodiments,
wherein the regression model is a linear regression model.

A034. The method of A033 or any one of the embodiments,
wherein the regression model is a logistic regression model.

A041. The method of any one of A001 to A034 or any one of the embodiments,
wherein the diagnostic data comprises gene expression levels.

A042. The method of any one of A001 to A034 or any one of the embodiments,
wherein the diagnostic data comprises RNA expression levels.

A043. The method of A042 or any one of the embodiments,
wherein the RNA expression levels are acquired by a microarray measurement or a sequencing method on RNAs.

A044. The method of A043 or any one of the embodiments,
wherein the RNA expression levels comprise a profile including a plurality of RNA expression levels.

A045. The method of any one of A042 to A044 or any one of the embodiments,
wherein the RNA may be cfRNA, RNA in cells, or RNAs included in extracellular vesicles.

A046. The method of A045 or any one of the embodiments,
wherein the RNA is selected from the group consisting of miRNA, and mRNA.

A047. The method of any one of A043 to A046 or any one of the embodiments,
wherein the RNA is derived from a body fluid selected from the group consisting of: blood, serum, plasma, lymph fluid, tissue fluids, interstitial fluid, intercellular fluid, cavity fluid, serosal fluid, pleural fluid, ascites fluid, pericardial fluid, cerebrospinal fluid, joint fluid (synovial fluid), and aqueous humor of the eye (aqueous).

A048. The method of any one of A043 to A046 or any one of the embodiments,
wherein the RNA is derived from a tissue obtained by a biopsy or during a surgical operation.

A051. The method of any one of A001 to A048 or any one of the embodiments,
wherein the disease is a cancer.

A052. The method of A051 or any one of the embodiments,
wherein the cancer is selected from a group of: brain tumor, lung cancer, breast cancer, thyroid cancer, esophagus cancer, liver cancer, biliary tract cancer, gastric cancer, pancreas cancer, colorectal cancer, prostate cancer, renal cancer, bladder cancer, uterine cancer, cervical cancer, ovarian cancer, skin cancer, lymphoma, leukemia.

B001. A method of predicting a disease status of a patient, to be executed by a processor, the method comprising:
a) training a classifier to predict a disease status of a patient, comprising:
   i) receiving a plurality of training data sets derived from a plurality of cohorts, each training data set comprising: a diagnostic data; a cohort ID; and a disease ID;
   ii) using the received plurality of training data sets to generate a batch classifier (having a plurality of batch IDs); and
   iii) using the received plurality of training data and the generated cohort classifier to generate a disease status classifier;
b) providing a patient data comprising a diagnostic data related to the patient;
c) using the generated batch classifier to select a batch ID among the plurality of batches, which the patient data is likely to match among the plurality of batches; and
d) using the selected cohort ID and the patient data to predict a disease status of the patient.

B001b. A method of predicting a disease status of a patient, to be executed by a processor, the method comprising:
a) training a classifier to predict a disease status of a patient, comprising a method of any one of A001 to A052;
b) providing a patient data comprising a diagnostic data related to the patient;
c) using the generated cohort classifier to select a cohort ID among the plurality of cohorts, which the patient data is likely to match among the plurality of cohorts; and
d) using the selected cohort ID and the patient data to predict a disease status of the patient.

B002. A method of predicting a disease status of a patient, to be executed by a processor, the method comprising:
a) providing a classifier to predict a disease status of a patient, the classifier being trained by:
   i) receiving a plurality of training data sets derived from a plurality of batches, each training data set comprising: a diagnostic data; a batch ID; and a disease ID;
   ii) using the received plurality of training data sets to generate a batch classifier; and
   iii) using the received plurality of training data and the generated batch classifier to generate a disease status classifier;
b) providing a patient data comprising a diagnostic data related to the patient;
c) using the generated cohort classifier to select a cohort ID among the plurality of cohorts, which the patient data is likely to match among the plurality of cohorts; and
d) using the selected cohort ID and the patient data to predict a disease status of the patient.

B003. A method of predicting a disease status of a patient, to be executed by a processor, the method comprising:
a) providing a trained disease status classifier comprising:
   a main neural network architecture having an input layer, at least one hidden layers, and an output layer, to predict a disease status of a patient; and
   a batch classifier having a list of batch IDs, the batch classifier being configured to output to one of the at least one hidden layers;
b) inputting a patient data comprising a diagnostic data related to the patient, to the disease status classifier;
c) using the generated batch classifier to select a batch ID, which the patient data is likely to match among the list of batches; and
d) using the selected batch ID and the patient data to output a disease status of the patient from the output layer.

C001. A computer program, to be executed by a processor, comprising a method of any one of B001 to B003.

D001.

A computer readable storage medium comprising a computer program of C001. E001. A computer system of classifying a disease status of a patient, comprising:
at least one processor; and
a memory storing at least one program to be executed by the at least one processor, the program comprising at least one of A001 to A052 and B001 to B003.

While several embodiments and examples of the present disclosure have been described above, these embodiments and examples illustrate the present disclosure. For example, each of the embodiments described above has been described in detail in order to explain the present disclosure in an easy-to-understand manner, and dimensions, configurations, materials, and circuits may be additionally changed as necessary. Embodiments in which one or more features of the present disclosure described above are arbitrarily combined are also included in the scope of the present disclosure. It is intended that the appended claims cover numerous modifications to the embodiments without departing from the spirit and scope of the present disclosure. Accordingly, the embodiments and examples disclosed herein have been shown by way of examples and should not be considered as limiting the scope of the present disclosure.

### Citations

Leek, Jeffrey T.; Johnson, W. Evan; Parker, Hilary S.; Jaffe, Andrew E.; Storey, John D. (2012-03-15). “The sva package for removing batch effects and other unwanted variation in high-throughput experiments”. Bioinformatics. 28 (6): 882-883.

1. A method of training a classifier to predict a disease status of a patient, to be executed by a processor, the method comprising:
i) receiving a plurality of training data sets derived from a plurality of batches, each training data set comprising: a diagnostic data; a batch ID; and a disease ID;
ii) using the received plurality of training data sets to generate a batch classifier; and
iii) using the received plurality of training data and the generated batch classifier to generate a disease status classifier.

2. A method of training a classifier to predict a responsible variable of a subject, to be executed by a processor, the method comprising:
i) receiving a plurality of training data sets derived from a plurality of batches, each training data set comprising: an (one or more) explanatory variable; a batch ID; and a target variable;
ii) using the received plurality of training data sets to generate a batch classifier; and
iii) using the received plurality of training data and the generated batch classifier to generate a target variable classifier.

3. The method of claim 1,
wherein said generating the disease status classifier comprises using a neural network.

4. The method of claim 3,
wherein the neural network comprises an input layer, at least one hidden layers and an output layer, and
wherein iii) comprises inputting an output of the batch classifier in one of the at least one hidden layers.

5. The method of claim 3,
wherein the neural network comprises an input layer, a plurality of hidden layers and an output layer, and
wherein iii) comprises inputting an output of the batch classifier in one of the plurality of hidden layers.

6. The method of claim 5,
wherein iii) comprises inputting an output of the batch classifier in one of the second half from the middle of the plurality of hidden layers.

7. The method of claim 5,
wherein iii) comprises inputting an output of the batch classifier in one of the last third of the plurality of hidden layers.

8. The method of claim 5,
wherein iii) comprises inputting an output of the batch classifier in one of the last quarter of the plurality of hidden layers.

9. The method of claim 5,
wherein iii) comprises inputting an output of the batch classifier in the last hidden layer just before the output layer.

10. The method of claim 4,
wherein the neural network is selected from the group consisting of: Perceptron (P), Feed Forward (FF), Radial Basis Function Network (RBF), Deep Feed Forward (DFF), Recurrent Neural Network (RNN), Long/Short Term Memory (LSTM), Gated Recurrent Unit (GRU), Auto Encoder (AE), Variational AE (VAE), Denoising Auto Encoder (DAE), Sparse AE (SAE), Markov Chain (MC), Hopfield Network (HN), Boltzmann Machine (BM), Restricted BM (RBM), Deep Belief Network (DBN), Deep Convolutional Network (DCN), Deconvolutional Network (DN), Deep Convolutional Inverse Graphics Network (DCIGN), Generative Adversarial Network (GAN), Liquid State Machine (LSM), Extreme Learning Machine (ELM), Echo State Network (ESN), Deep Residual Network (DRN), Kohonen Network (KN), Support Vector Machine (SVM), and Neural Turing Machine (NTM).

11. The method of claim 1,
wherein ii) comprises training the batch classifier.

12. The method of claim 11,
wherein said training the batch classifier comprises using a regression model.

13. The method of claim 12,
wherein the regression model is a linear regression model.

14. The method of claim 13,
wherein the regression model is a logistic regression model.

15. The method of claim 1,
wherein the diagnostic data comprises gene expression levels.

16. The method of claim 1,
wherein the diagnostic data comprises RNA expression levels.

17. The method of claim 16,
wherein the RNA expression levels are acquired by a microarray measurement or a sequencing method on RNAs.

18. The method of claim 17,
wherein the RNA expression levels comprise a profile including a plurality of RNA expression levels.

19. The method of claim 16,
wherein the RNA may be cfRNA, RNA in cells, or RNAs included in extracellular vesicles.

20. The method of claim 19,
wherein the RNA is selected from the group consisting of miRNA, and mRNA.

21. The method of claim 17,
wherein the RNA is derived from a body fluid selected from the group consisting of: blood, serum, plasma, lymph fluid, tissue fluids, interstitial fluid, intercellular fluid, cavity fluid, serosal fluid, pleural fluid, ascites fluid, pericardial fluid, cerebrospinal fluid, joint fluid (synovial fluid), and aqueous humor of the eye (aqueous).

22. The method of claim 17,
wherein the RNA is derived from a tissue obtained by a biopsy or during a surgical operation.

23. The method of claim 1,
wherein the disease is a cancer.

24. The method of claim 23,
wherein the cancer is selected from a group of: brain tumor, lung cancer, breast cancer, thyroid cancer, esophagus cancer, liver cancer, biliary tract cancer, gastric cancer, pancreas cancer, colorectal cancer, prostate cancer, renal cancer, bladder cancer, uterine cancer, cervical cancer, ovarian cancer, skin cancer, lymphoma, leukemia.

25. A method of predicting a disease status of a patient, to be executed by a processor, the method comprising:
a) training a classifier to predict a disease status of a patient, comprising:
i) receiving a plurality of training data sets derived from a plurality of batches each training data set comprising: a diagnostic data; a batch ID; and a disease ID;
ii) using the received plurality of training data sets to generate a batch classifier (having a plurality of batch IDs); and
iii) using the received plurality of training data and the generated batch classifier to generate a disease status classifier;
b) providing a patient data comprising a diagnostic data related to the patient;
c) using the generated batch classifier to select a batch ID among the plurality of batches, which the patient data is likely to match among the plurality of batches; and
d) using the selected batch ID and the patient data to predict a disease status of the patient.

26. A method of predicting a disease status of a patient, to be executed by a processor, the method comprising:
a) training a classifier to predict a disease status of a patient, comprising the method of claim ;
b) providing a patient data comprising a diagnostic data related to the patient;
c) using the generated batch classifier to select a batch ID among the plurality of batches, which the patient data is likely to match among the plurality of batches; and
d) using the selected batch ID and the patient data to predict a disease status of the patient.

27. A method of predicting a disease status of a patient, to be executed by a processor, the method comprising:
a) providing a classifier to predict a disease status of a patient, the classifier being trained by:
i) receiving a plurality of training data sets derived from a plurality of batches, each training data set comprising: a diagnostic data; a batch ID; and a disease ID;
ii) using the received plurality of training data sets to generate a batch classifier; and
iii) using the received plurality of training data and the generated batch classifier to generate a disease status classifier;
b) providing a patient data comprising a diagnostic data related to the patient;
c) using the generated batch classifier to select a batch ID among the plurality of batches, which the patient data is likely to match among the plurality of batches; and
d) using the selected batch ID and the patient data to predict a disease status of the patient.

28. A method of predicting a disease status of a patient, to be executed by a processor, the method comprising:
a) providing a trained disease status classifier comprising:
a main neural network architecture having an input layer, at least one hidden layers, and an output layer, to predict a disease status of a patient; and
a batch classifier having a list of batch IDs, the batch classifier being configured to output to one of the at least one hidden layers;
b) inputting a patient data comprising a diagnostic data related to the patient, to the disease status classifier;
c) using the generated batch classifier to select a batch ID, which the patient data is likely to match among the list of batches;
d) using the selected batch ID and the patient data to output a disease status of the patient from the output layer.

29. A computer program, to be executed by a processor, comprising the method of claim 26.

30. A computer system of classifying a disease status of a patient, comprising:
at least one processor; and
a memory storing at least one program to be executed by the at least one processor, the program comprising the method of claim 1.
